(19) United States
(12) Patent Application Publication
(10) Pub. No.: US 2021/0228911 A1
(43) Pub. Date: Jul. 29, 2021

(54) BEAM ENERGY MEASUREMENT SYSTEM

(71) Applicants: ADAM S.A., Meyrin (CH); UNIVERSITA' DEGLI STUDI DI BERGAMO, Bergamo (IT)

(72) Inventors: Michele CALDARA, Geneva (CH); Francesco GALIZZI, San Pellegrino Terme, Bergamo (IT); Adam JEFF, Ferney Voltaire (FR)

(73) Assignees: ADAM S.A., Meyrin (CH); UNIVERSITA' DEGLI STUDI DI BERGAMO, Bergamo (IT)

(21) Appl. No.: 17/046,773

(22) PCT Filed: Apr. 12, 2019
(86) PCT No.: PCT/EP2019/059376

§ 371 Date: Oct. 10, 2020

(30) Foreign Application Priority Data
Apr. 13, 2018 (EP) 18167210.6

(51) Int. Cl. A61N 5/10; G01T 1/29; H05H 9/04

(52) U.S. Cl. CPC A61N 5/1077; G01T 1/2914; A61N 2005/1087; H05H 2277/11; H05H 9/041

(57) ABSTRACT

A time-of-fight measurement system for measuring energy of a pulsed hadron beam, wherein each pulse of the beam is structured into a series of bunches of charged particles, said bunches being repeated according to a repetition rate of the order of magnitude of radiofrequency. The system comprises a first detector, a second detector and a third detector arranged along a beam path, each of the detectors being configured to detect the passage of a bunch of charged particles and provide an output signal dependent on phase of the detected bunch, wherein the second detector is spaced apart from the first detector by a first distance and wherein the third detector is spaced apart from the second detector by a second distance, wherein the first distance is set out in such a way as that time of flight of the bunch from the first detector to the second detector is approximately equal to, or lower than a repetition period of the bunches, and wherein the second distance is set out in such a way as that time of flight of the bunch from the second detector to the third detector is greater than a multiple of the repetition period of the bunches, and a processing unit configured to a) calculate phase shifts between the output signals of the detectors, and b) calculate energy of the pulse based on the calculated phase shifts.

### BACKGROUND OF THE INVENTION

The present invention relates generally to beam energy measurement systems for particle accelerators.

Linear accelerators are used are used in radiotherapy to accelerate particles, typically electrons, protons or heavier ions including helium or carbon ions, up to energies sufficient to allow them to travel to a depth in tissue to irradiate and impart energy to a tumor. In the case of electrons they may alternatively be directed onto a target of material of large atomic number to create high energy X-rays which themselves are then used to treat a tumor at depth.

Typically hadronic and ion particles are generated in a source (for example an Electron Cyclotron Resonance Ion Source (ECRIS) or ion plasma source for protons) and injected into a linear accelerator complex where they are accelerated by high frequency radiofrequency (RF) fields up to a required energy or energies. Acceleration typically proceeds in stages, which may include a pre-accelerator stage, for example a radiofrequency quadrupole (RFQ). The production of a high energy output beam, suitable for radiotherapy treatment or other use, in practice may involve several accelerator sub-units, possibly as many as 10-14, each comprising a sequence of individual accelerator cavities connected to waveguides arranged to couple in the driving RF fields. Typical accelerator stages include drift tube linacs (DTLs), side coupled drift tube linacs (SCDTLs) and coupled cavity linacs (CCLs). The RF fields are typically produced by klystrons or magnetrons.

Typically between the source and pre-accelerator stage is a low energy beam transfer line (LEBT). Typically a medium energy beam transfer line (MEBT) is situated between each accelerator sub-unit or between groups of sub-units. The beam path from beginning to end of the accelerator complex may be many meters long and is typically shielded throughout its length.

In one linear accelerator solution a proton beam is formed into pulses in a proton source injector assembly and these are introduced into a pre-accelerator stage, typically a Radiofrequency Quadrupole (or RFQ) which accelerates the initially drifting pulses up to 5 MeV. During this process of pre-acceleration the pulses gain a bunched structure at 750 MHz as the protons in the pulse start to interact with the accelerating RF field. At the output of the RFQ each pulse is fed into the input of a first linear accelerator stage as a bunched pulse, for eventual acceleration up to medically useful treatment energies. In a particular embodiment a chopper element is arranged to create the beam pulse in a proton source injector assembly. The chopper element, the pre-accelerator stage and the linear accelerator stages operate at a repetition rate of up to 200 Hz. In each subsequent linear accelerator stage applied RF fields couple to the bunches in each pulse and accelerate them to higher and higher energies, while maintaining the structure of the pulse.

The final output energy of the beam from a linear accelerator will be dependent on the number of accelerating structures that are present and the maximum energy is typically equal to the maximum possible energy to which particles can be accelerated. However, it is also possible to vary the energy at output by switching off active units at the end of the linear accelerator gallery. The energy of the emerging beam at output is then equal to the output energy of the last active accelerating unit.

In medical particle accelerators the beam which is produced at the output of the last accelerating sub-unit is transported to the patient through a high energy beam transfer line (HEBT). At the end of the HEBT is a nozzle which is typically arranged to direct, or scan the beam, at the target in the patient, and which nozzle typically also includes a dose delivery system arranged to monitor the dose delivered to the patient.

In a synchrotron based medical accelerator the beam energy necessary for the tumor slice treatment is achieved with a combination of settings in the synchrotron accelerating cavity and in the dipoles' magnetic field, while in cyclotron-based machines it is reached with the insertion of material in the beam by means of the energy degrader in the HEBT. In both cases there is no necessity to accurately measure the energy of the beam directed to the patient, since it can be assumed that it is the same obtained during machine commissioning or quality assurance phase.

Beam profile, beam current and—especially in the Linac-based proton therapy accelerator with variation of energy—beam energy are typically monitored for beam diagnostics or clinical purposes and a beam energy measurement system is typically mandatory under medical device regulations. The HEBT typically has an acceptance in beam energy much higher than the treatment requirements and this could result in a beam with slightly different, or even higher, energy from that which was requested being transported to the patient.

The Dose Delivery System, installed just before the patient, has no means of measuring beam energy of the pulses delivered to the patient. Compliance with medical device regulations may require that the system should incorporate energy measurement for every beam pulse delivered to the patient.

A classical method to measure beam energy is a spectrometer-based system, which includes the use of a bending dipole in the HEBT, in combination with beam position and/or profile detectors upstream and downstream. In case the HEBT is straight, this method cannot be used.

In view of the above, there is a need for a fast response beam energy measurement system. Furthermore, there is a need for a beam energy measurement system which is not affected by the layout of the beam transfer lines.

### SUMMARY OF THE INVENTION

Accordingly, there is provided a time-of-flight (TOF) measurement system for measuring energy of a pulsed hadron beam, wherein each pulse of the beam is structured into a series of bunches of charged particles, said bunches being repeated according to a repetition rate of the order of magnitude of radiofrequency, said system comprising
a first detector, a second detector and a third detector arranged along a beam path, each of said detectors being configured to detect the passage of a hunch of charged particles and provide an output signal dependent on phase of the detected bunch, wherein the second detector is spaced apart from the first detector by a first distance and wherein the third detector is spaced apart from the second detector by a second distance, wherein said first distance is set out in such a way as that time of flight of the bunch from the first detector to the second detector is equal to, or lower than a repetition period of the bunches, and wherein said second distance is set out in such a way as that time of flight of the bunch from the second detector to the third detector is greater than a multiple of the repetition period of the bunches, and
processing means configured to

a) calculate phase shifts between the output signals of the detectors, and

b) calculate energy of the pulse based on the calculated phase shifts.

According to an embodiment, there is provided a time-of-flight (TOF) measurement system for measuring energy of a pulsed hadron beam, wherein each pulse of the beam is structured into a series of bunches of charged particles, said bunches being repeated according to a repetition rate of the order of magnitude of radiofrequency, said system comprising
a first detector, a second detector and a third detector arranged along a beam path, each of said detectors being configured to detect the passage of a bunch of charged particles and provide an output signal dependent on phase of the detected bunch, wherein the second detector is spaced apart from the first detector by a first distance and wherein the third detector is spaced apart from the second detector by a second distance, wherein said first distance is set out in such a way that the difference between the time of flight from the first detector to the second detector for bunches of the highest and lowest energies accepted by the HEBT is equal to or lower than the repetition period of the bunches, and wherein said second distance is set out in such a way as that time of flight of the bunch from the second detector to the third detector is greater than a multiple of the repetition period of the bunches, and
processing means configured to

a) calculate phase shifts between the output signals of the detectors, and

b) calculate energy of the pulse based on the calculated phase shifts.

The bunches can potentially have a very high repetition rate (up to 3 GHz). The signal strength typically depends on beam intensity as well as beam energy and can vary within a very broad range (for example, more than 3 orders of magnitude). As an example it may vary from −60 dBm to 7 dBm. The system can measure a beam pulse average energy within a range from a minimum energy of, for example, 5 MeV to a maximum energy of, for example, 230 MeV. The system is not interceptive and can be used with any kind of hadron.

Advantageously, the beam energy measurement system according to the invention may be used in a control system of a particle accelerator for radiotherapy, allowing a pulse by pulse control or monitoring of the beam energy; this means that if the energy of a pulse varies from a requested energy by a certain extent (for example by, say, 0.17%) then the next pulse can be prevented or stopped, or mitigated in some way so that it is not delivered to the patient.

More in general, the system of the invention allows a much higher bunches repetition rate than conventional systems. Current systems do not reach 400 MHz; this system can potentially operate up to 1 GHz or higher.

Furthermore, the system allows a very high measurement repetition rate (up to 200 Hz). Moreover, it has an energy detection accuracy, which may in some embodiments be as high as 0.03%, which makes it usable in the Beam Delivery System.

For example, it may be that a medical system (with maximum energy of 230 MeV) is legally required to be able to measure beam E with a resolution of mm water equivalent at maximum energy (230 MeV). This is a challenging case for the ToF system and is equivalent of having 0.15% beam energy resolution. For this reason in an embodiment it was decided to fix the energy resolution requirement to be 5 times better than a possible legal requirement, thus 0.03% across the beam energy range.

The invention is independent of the layout of the transfer lines, and since it does not comprise a spectrometer it can be used in both straight and curved transfer lines, and can detect fast energy changes in both. However it is most advantageously used in a straight transfer line. In fact it may be installed in any straight sector of the machine, and in particular after the pre-accelerator or RFQ which provides bunching.

The invention is advantageously situated in the HEBT where it may be used to measure the output energy of the proton pulses.

### BRIEF DESCRIPTION OF THE DRAWINGS

Some preferred, but non-limiting, embodiments of the invention will now be described, with reference to the attached drawings, in which:

FIG. 1 shows a diagram of a detector arrangement of a beam energy measurement system according to the invention,

FIG. 2 shows a graph reporting limits on the distance and phase-shift errors for achieving 0.03% precision in the energy measurement at E=230 MeV,

FIG. 3 shows a hardware design of a prototype system according to the invention,

FIGS. 4 to 9 show flow diagrams of an exemplary beam energy measurement method, and

FIG. 10 shows a HEBT in which the invention may be situated.

### DETAILED DESCRIPTION

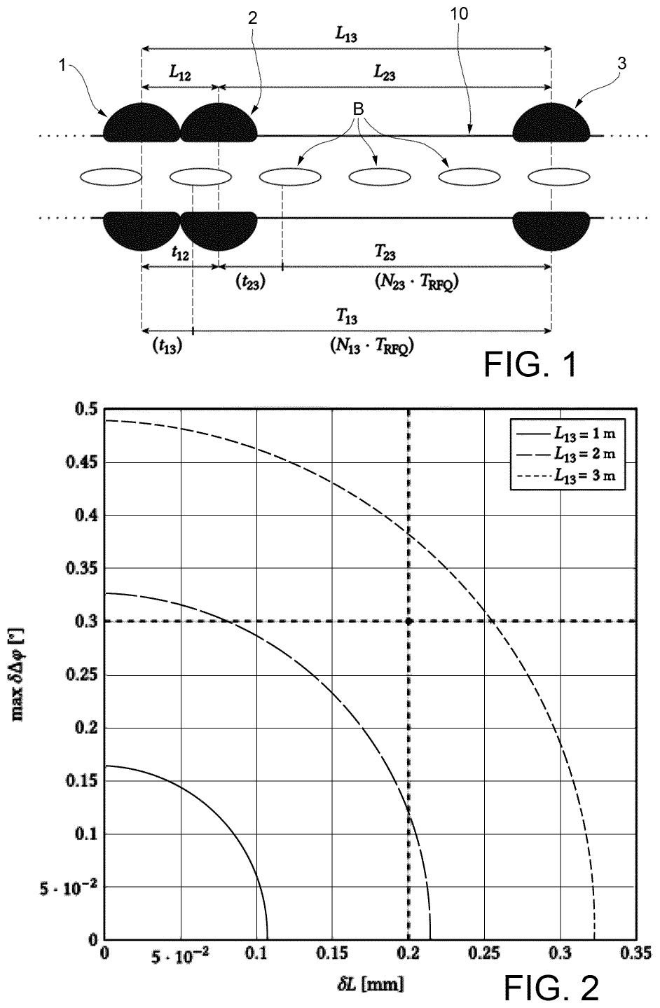

With reference to FIG. 1, the energy measurement system according to the invention comprises a first detector 1, a second detector 2 and a third detector 3 arranged along a beam path 10, for example along a beam pipe of an accelerator HEBT. Each of the detectors 1, 2, 3 is configured to detect the passage of a bunch of charged particles and provide an output signal dependent on the phase of the detected bunch. Beam bunches are designated with B in FIG. 1. Each separate train of bunches constitutes a pulse. The second detector 2 is spaced apart from the first detector 1 by a first distance L₁₂. The third detector 3 is spaced apart from the second detector 2 by a second distance L₂₃. Distance between the first detector 1 and the third detector 3 is designated with L₁₃.

In an embodiment the detectors 1-3 are capacitive pickups and in a specific embodiment these are phase probes. In place of phase probes a beam position monitor, a beam current transformer or a wall current monitor might be used. Alternatively a resonant cavity or an electro-optic crystal may be used. In general any device which measures the electric or magnetic fields of the particle beam is suitable. In an alternative embodiment a beam loss monitor or a device that intercepts part of the beam halo could be used.

Phase probes are capacitive sensors that can be used to detect in a non-interceptive way the passage of a bunch of charged particles. Their main component is a metallic ring, placed around the beam or beam pipe, on which a charge develops when a beam bunch passes inside it. This charge can be collected to get a current proportional to the variation of charge inside the ring.

t₁₂ is the time taken by a particle bunch B to travel the distance L₁₂, which can be used to compute the particles energy:

$$β = \frac{{L}_{12}}{{t}_{12}} \cdot \frac{1}{c}$$

$$γ = \frac{1}{\sqrt{1 - {β}^{2}}}$$

$$E = {E}_{0} \cdot ( γ - 1 )$$

where E is the kinetic energy of the particle and E₀ is the rest energy of the particle (for protons: E₀=938.272 MeV); c is the speed of light.

The system is designed to measure the phase shift Δϕ between the output signal of the probes 1-3. To be able to compute t₁₂ from Δϕ₁₂ (i.e. the phase shift between the output signal of the first detector 1 and the output signal of the second detector 2) the relation between the two has to be unambiguous. To this end, the first distance L₁₂ is set out in such a way that the time of flight t₁₂ of a bunch B from the first detector 1 to the second detector 2 is equal to, or lower than a repetition period T_{RFQ} of the bunches B. This poses a limit on the maximum value of L₁₂. For an energy range from 5 MeV to 230 MeV this limit is around 48 mm.

In a particular example the detectors of the invention are situated in a HEBT layout with distance L₁₂=255 mm and distance L₁₃=3595 mm. These distances provide a 0.03% E resolution for beams ranging from 70 up to 230 MeV, as shown in FIG. 2.

Given the limit on L₁₂, it is impossible to achieve 0.03% of relative error on the measurement of E with only two close probes, having only one bunch travelling through them, because this would require a precision in the phase shift measurement which is nowadays unachievable. This is the reason behind the use of a third probe. L₂₃ is much greater than L₁₂, so more than one bunch can be positioned along L₂₃. In other words, time of flight T₂ of the bunch from the second detector 2 to the third detector 3 is greater than a multiple of the repetition period T_{RFQ} of the bunches. N₁₃ and N₂₃ represent the number of whole bunches present between, respectively, detectors 1 and 3, 2 and 3. In an embodiment, the repetition rate T_{RFQ} is given by the RFQ of the linear accelerator. Note that using only two distant probes is not sufficient as this would not allow an unambiguous energy measurement over the range from 5 MeV to 230 MeV. This is because using the two distant probes only (for example 1 and 3 of FIG. 1) the train of bunches (typically around 1000 of them into a beam pulse) passing through the phase probes, induces a train of signals reaching the acquisition system in the same acquisition window (typically 1 microsecond). Excluding the tails of the bunched pulse (affected by noise), it is not possible to recognize which is the integer number of bunches to skip before computing the ToF (N₁₃ of FIG. 1.). We could for example in this instance assume N₁₃+1 or N₁₃−1, resulting then in a wrong measurement of energy. If instead an extra detector between detectors 1 and 3 of FIG. 1 is included as previously explained, it is possible to determine the approximate energy and thus N₁₃.

Expressed in another way, if only two detectors were used placed so closely together that only one bunch was present in the inter-detector beamline at any one instant, then the measurement made by both detectors could be interpreted unambiguously as the measurement of the same bunch. However the measurement made, while unambiguous, would be inaccurate because of the phase difference. In fact the real ToF between the two extremely closely spaced detectors would be extremely small and the relative error would be large (calculated from error in measurement=error of instrument/distance between detectors). If we increase the distance between the two detectors so that more than one bunch can now fit simultaneously between the two detectors then the measurement will now result in the detection of two trains of bunches, shifted from each other, and it will not be possible to predict which detected bunch in each train should become the basis of the energy measurement, or in other words how many bunches N should be skipped. To overcome this problem, i.e. to know the correct value of N, it is necessary to already know the energy of the bunches, but calculating energy is the point of the measurement so we face a conundrum.

As an example of this, if the energy of the beam is 100 MeV and:

L₁₂=225 mm,

L₁₃=3595 mm,

delta_L=0.1 mm, and

delta_phi=0.2 deg

implies:

energy_error_12=0.12%,

energy_error_13=0.01%

In an alternative embodiment, if:

L₁₂=40 mm,

L₁₃=1000 mm,

delta_L=0.1 mm, and

delta_phi=0.2 deg

implies:

energy_error_12=0.7%,

energy_error_13=0.025%

However, a worst case occurs if the energy is 230 MeV (a maximum energy in some systems), in which case the corresponding error values are:

in the first case.

energy_error_12 is 0.21% (instead of 0.12%),

energy_error_13 is still 0.01%;

in the second case,

energy_error_12 is 1.16% (instead of 0.7%),

energy_error_13 is 0.05% (instead of 0.025%).

Therefore we measure the approximate beam energy using the signals from detectors 1 and 2 (which allow unambiguous but inaccurate calculation of energy) and use this approximation to calculate the number N of bunches which must be skipped to allow an unambiguous measurement between detectors 1 and 3 (which suffer from ambiguity but provide for a more accurate calculation of energy). By doing this we simultaneously reduce inaccuracy while maintaining unambiguity of calculation. Therefore three detectors are needed to produce a measurement which is both unambiguous and accurate.

This layout greatly improves the energy measurement precision; given the precision in the distance measurement δL and in the phase shift measurement δΔϕ, the relative error on the energy measurement using only detectors 1 and 2 is

$$\frac{{β}^{2} {γ}^{3}}{γ - 1} \cdot \sqrt{{( \frac{δ L}{{L}_{12}} )}^{2} + {( \frac{δ Δφ}{2 π} \cdot \frac{{T}_{RFQ}}{{t}_{12}} )}^{2}}$$

While when using also detector 3 it is

$$\frac{{β}^{2} {γ}^{3}}{γ - 1} \cdot \sqrt{{( \frac{δ L}{{L}_{13}} )}^{2} + {( \frac{δ Δφ}{2 π} \cdot \frac{{T}_{RFQ}}{{t}_{13}} )}^{2}}$$

which can be reduced by increasing L₁₃ (T₁₃ also increases consequently). The same reasoning can be applied to the opposite situation, i.e. in the case in which they are arranged in ‘reverse order’, and in this case the distance L₂₃ is set out in such a way as that time of flight t₂₃ of the bunch B from the second detector 2 to the third detector 3 is equal to, or lower than a repetition period T_{RFQ} of the bunches B, and then having L₁₂ much greater than L₂₃ such that the time of flight T₁₂ of the bunch from the first detector 1 to the second detector 2 is greater than a multiple of the repetition period T_{RFQ} of the bunches. In such a case N₂₃ will always be 0 and N₁₂ has to be used in its place.

FIG. 2 shows another important aspect of the previous formulas; in the design phase of the accelerator layout the distance L₁₃ should be chosen in such way that the measurement errors on both the phase and the distance still allow to achieve the beam energy resolution needed by a medical accelerator. δΔϕ is given by the electronics and from eventual interferences from high power sources in the accelerator, while δL is the result of the accuracy of survey instruments commonly used in the accelerator alignment, like a laser tracker (typical accuracy better than ±50 μm) combined with the uncertainty on the ring electrode alignment with respect to external references (typical accuracy ±50 μm). FIG. 2 depicts the upper limits on δL and δΔφ for achieving 0.03% precision in the energy measurement of a beam at 230 MeV at different values for L₁₃. The allowed combinations of δΔϕ and δL are those sitting below the curves, and the dot corresponding to the actual system performance is indicated.

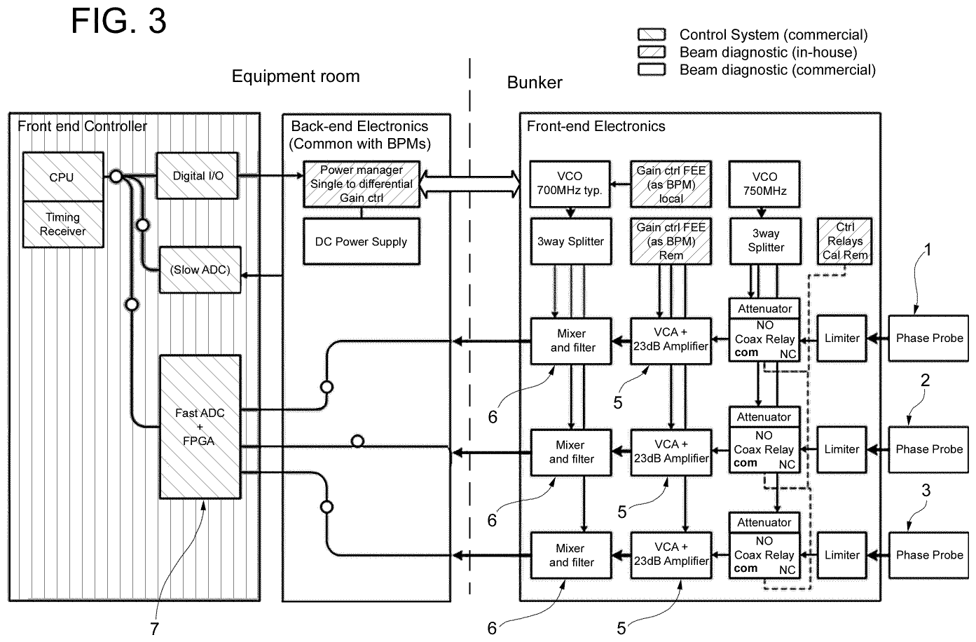

An example of hardware design which allows to measure the phase shifts between the output signals of the phase probes is shown in FIG. 3. The signals formed on the detectors 1, 2, 3 at beam passage enter limiters, devices used only to protect downstream electronics from unwanted spikes on the signals and successively they pass through RF coaxial relays, where they are routed to preamplification stages (at 5), where it is eventually possible to remotely change the gain. The amplified signals are then mixed down from 750 MHz to about 50 MHz (at 6), before being transmitted along coaxial cables that exit the machine room and enters directly a fast ADC plus FPGA (at 7) for acquisition and processing. The down-mix is not necessary, it is an implementation choice to relax requirements on the ADC and processing. The coaxial relays above mentioned are used to inject at the beginning of the three electronic chains a calibration signal at the same frequency of the bunches repetition rate (1/T_{RFQ}), so to acquire and subtract in the data processing the fixed contribution in phase offset between the three channels due to mismatches in the cable lengths and in the installed electronics blocks (amplifiers, mixers, etc). The calibration of the system should run only once for a few milliseconds and it can be repeated any time during ‘no beam’ operation (for example before a new treatment starts) so to eventually update the phase offset; in this way it is possible to compensate any kind of long-term effect on the system like temperature drifts in the accelerator room.

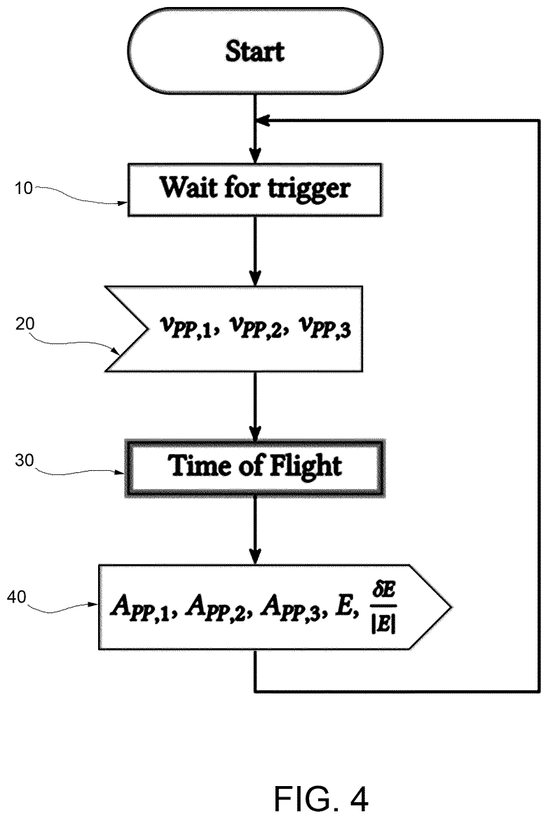

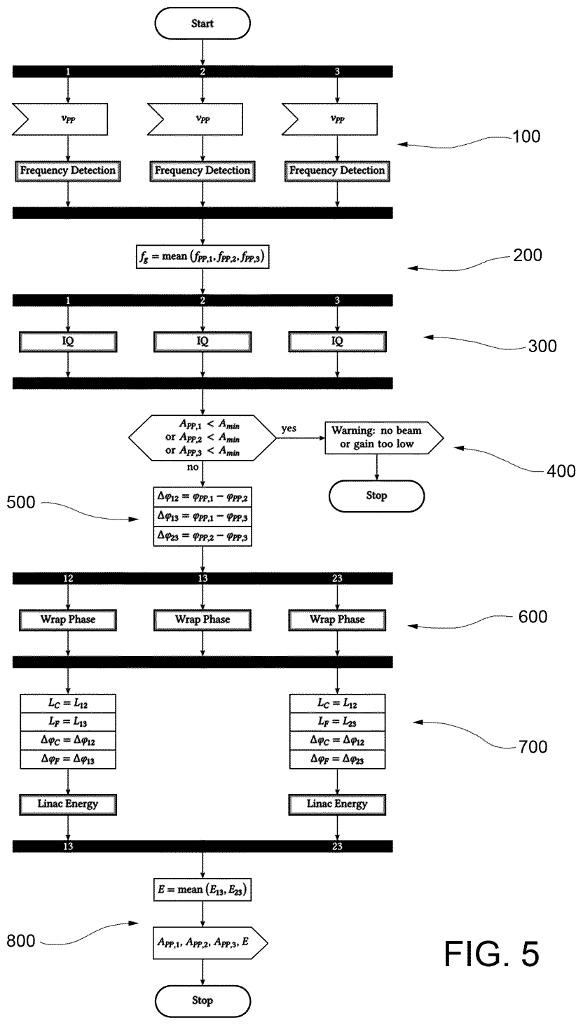

The diagrams in FIGS. 4-9 represent an example of the computation that takes place inside the FPGA 7, which extracts the energy information from the detectors signals. The algorithm can be resumed in the following steps (FIGS. 4 and 5):
Acquire the signals ν_{PP.1}, ν_{PP.2}, ν_{PP.3} coming from the detectors 1-3 (step 20). The exact time at which the acquisition should start (step 10 is computed based on a system-level trigger signal (which tells when the next pulse will occur). In a further embodiment it may also be based on the requirements that the detector signals are acquired for a duration which is slightly shorter than the pulse duration and starting slightly after the pulse so that the central part of the actual pulse is sampled.
Perform a time-of-flight analysis of the signals (step 30). As will be explained in the following, this analysis comprises:
   Detect the exact frequency f_{g} of the signals ν_{PP.1}, ν_{PP.2}, ν_{PP.3}. This is necessary because the down-mixing introduces some uncertainty in the signal frequency.
   Use the detected frequency f_{g} to perform an I/Q method on the signals, which gives the amplitude A_{PP.1}, A_{PP.2}, A_{PP.3} and the phase φ_{PP.1}, φ_{PP.2}, φ_{PP.3} of each signal.
   Check the signals amplitudes A_{PP.1}, A_{PP.2}, A_{PP.3} to see if the pulse has been correctly detected: If not, stop.
Use the signals phases φ_{PP.1}, φ_{PP.2}, φ_{PP.3} to compute the energy E, and broadcast this information across the control system (step 40).

In FIGS. 4-9, the following blocks are used:
[symbol] Start/Stop: Where the (sub)algorithm flow starts or stops.
[symbol] Input/Read/Acquire: n the main diagram, this means that a signal is acquired by the hardware. In sub-diagrams, this means that a variable that has previously been set by the caller is required for this sub-algorithm to work.
[symbol] Output/Write/Send: In the main diagram, this means that some kind of value is transmitted by the algorithm to some other processing unit that may be interested in that value. In sub-diagrams, this means that a variable that has been set by the sub-algorithm is made available to the caller.
[symbol] Computation: Perform some kind of computation.
[symbol] Decision/Branching: A point in the diagram where the algorithm flow can take different paths based on a predicate.
[symbol] Sub-diagram: Execute the specified sub-diagram.

Every variable should be set before it is used or alternatively it is set as an a priori known value; the latter is true for constants known from physics and for the following variables: Aₘᵢₙ: Minimum amplitude required for the beam pulse to be considered correctly detected. f_{sampling}: Sampling frequency.

A further explanation is required for the correct interpretation of the flow diagrams: Different flows enclosed by black horizontal lines represents operations that can be performed in parallel.

A label put at the top of a parallel branch has to be considered as additional subscript to every variable that appears in that branch, including input and output variables in sub-diagrams. If the label is at the bottom, only output variables gains the subscript.

With reference to FIG. 5, the frequencies f_{PP.1}, f_{PP.2}, f_{PP.3} of the output signals of the detectors 1-3 are detected (at 100).

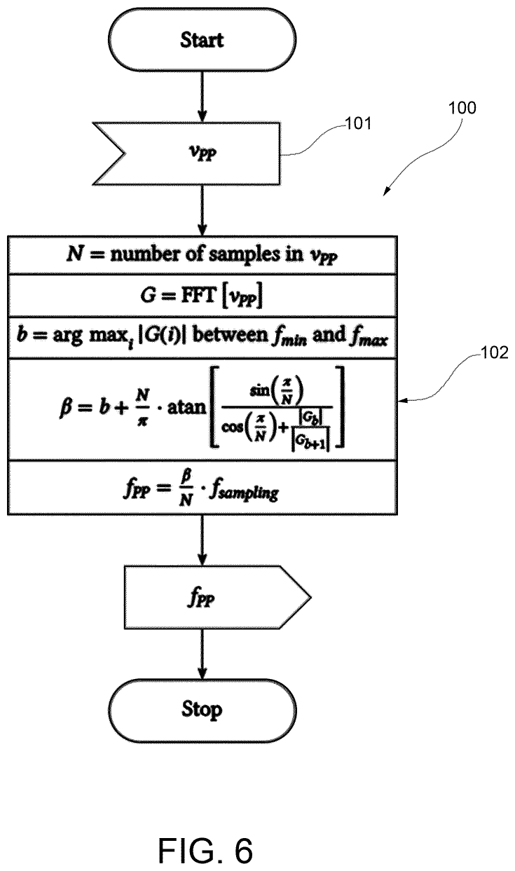

FIG. 6 show this detection procedure in detail. The output signal ν_{PP} of each detector is sampled with the sampling frequency f_{sampling} (at 101). Then, the following variables are

calculated at 102:

N=number of samples in ν_{PP}

G=Fast Fourier Transform of ν_{PP}.

b=arg maxᵢ|G(i)| between fₘᵢₙ and fₘₐₓ, wherein fₘᵢₙ and fₘₐₓ are minimum and maximum values which can be set to constrain the search for the maximum in the Fast Fourier Transform of ν_{PP}. They can be used when unknown frequencies are present in the Transform, although this should not be the normal situation.

$$β = b + \frac{N}{π} \cdot atan [ \frac{sin ( \frac{π}{N} )}{cos ( \frac{π}{N} ) + \frac{\vert {G}_{b} \vert}{\vert {G}_{b + 1} \vert}} ] .$$

Then, the frequency f_{PP} of each signal is calculated as

$${f}_{PP} = \frac{β}{N} \cdot {f}_{sampling} .$$

The frequency f_{g} of the signal is then calculated (at 200 in FIG. 5) as f_{g}=mean(f_{PP.1}, f_{PP.2}, f_{PP.3}).

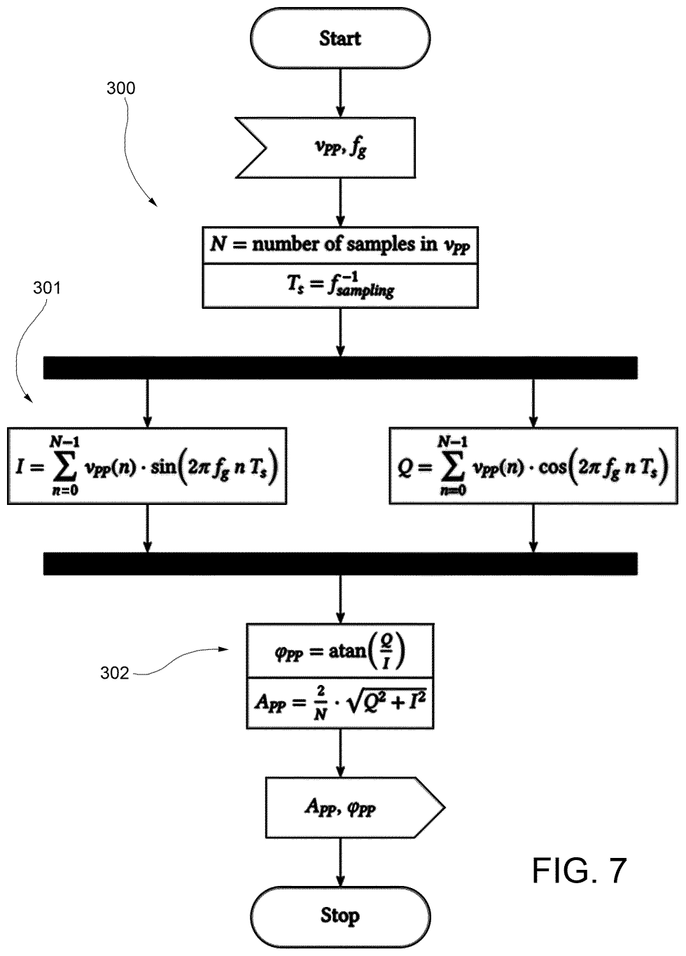

Then, an I/Q method is performed on each of the output signals ν_{PP} (at 300). The I/Q method is shown in detail in FIG. 7. The I and Q signals are calculated at 301 as

I=Σₙ₌₀^{N-1}ν_{PP}(n)·sin(2πf_{g}nTₛ),

Q=Σₙ₌₀^{N-1}ν_{PP}(n)·cos(2πf_{g}nTₛ).

where Tₛ=f¹_{sampling} is the sampling period.

Then, phase φ_{PP} and amplitude A_{PP} of each signal are calculated at 302 as

$${φ}_{PP} = atan ( \frac{Q}{I} ) , \\ {A}_{PP} = \frac{2}{N} \cdot \sqrt{{Q}^{2} + {I}^{2}} .$$

The signal amplitudes A_{PP} are then compared with Aₘᵢₙ (at 400 in FIG. 5). If at least one of these amplitudes is lower than Aₘᵢₙ, the process is stopped.

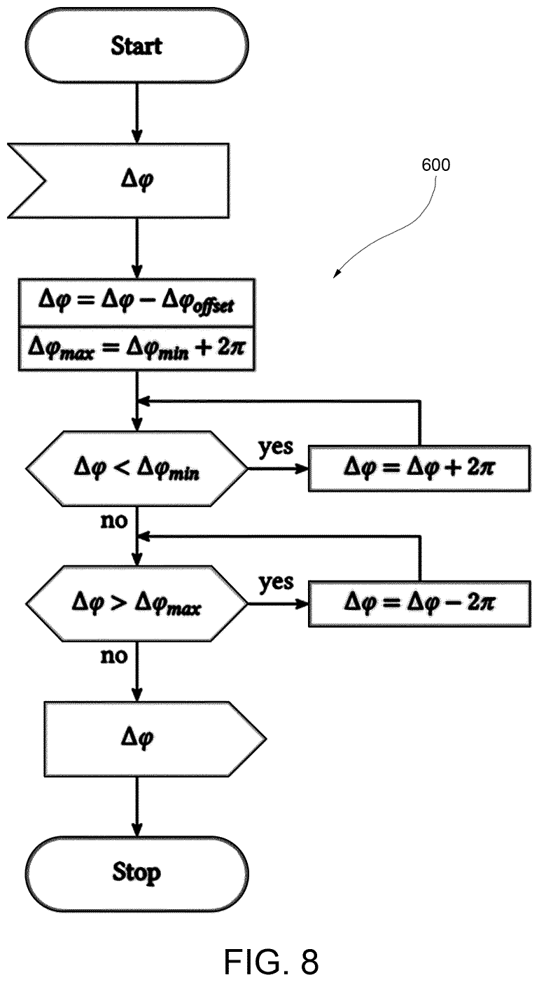

Otherwise, the phase shifts Δφ of the output signals are calculated at 500, and subjected to wrapping at 600 (see also FIG. 8).

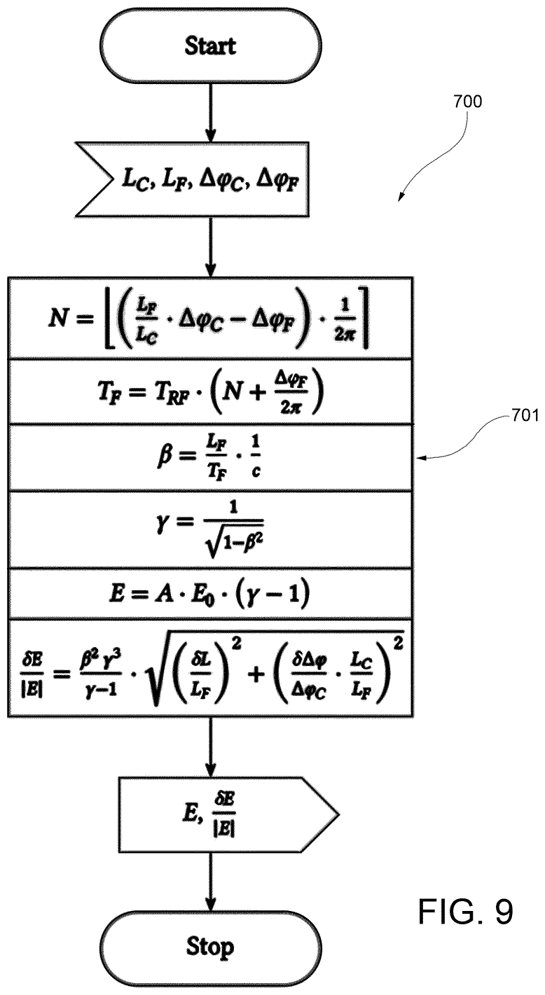

Then, energy values E₁₃ and E₂₃ of the pulse is calculated (at 700) based on time-of-flight measurements between detectors 1 and 3, and between detectors 2 and 3, respectively. This calculation is shown in detail in FIG. 9. The following variables are calculated at 701 (here, the subscript C takes place of subscript 12, while subscript F takes place of subscript 13 or 23, depending on whether E₁₃ or E₂₃ is calculated; T_{RF} corresponds to T_{RFQ} mentioned above):

$$N = [ ( \frac{{L}_{F}}{{L}_{C}} \cdot {Δφ}_{C} - {Δφ}_{F} ) \cdot \frac{1}{2 π} ] , \\ {T}_{F} = {T}_{RF} \cdot ( N + \frac{{Δφ}_{F}}{2 π} ) , \\ β = \frac{{L}_{F}}{{T}_{F}} \cdot \frac{1}{c} , \\ γ = \frac{1}{\sqrt{1 - {β}^{2}}} , \\ {E}_{( 13 or 23 )} = A \cdot {E}_{0} \cdot ( γ - 1 ) , \\ \frac{δ E}{\vert E \vert} = \frac{{β}^{2} {γ}^{3}}{γ - 1} \cdot \sqrt{{( \frac{δ L}{{L}_{F}} )}^{2} + {( \frac{δΔ φ}{{Δφ}_{C}} \cdot \frac{{L}_{C}}{{L}_{F}} )}^{2}}$$

The energy E of the pulse is calculated as a mean value between E₁₃ and E₂₃ (at 800).

According to an alternative embodiment, it would be sufficient to use E₁₃ or E₂₃ to provide the beam energy. The mean value between E₁₃ and E₂₃ is used to improve the measurement accuracy. The accuracy might be even further improved using a fourth detector/phase probe or more, but this would add complexity to the system.

The ToF beam energy measurement system allows a high accuracy beam energy measurement to be made at a very high measurement rate (up to 200 Hz) and provides the result typically within 1 ms from the passage of the beam pulse, making it suitable to be used not only as Beam Diagnostics device but also in the Beam Delivery System to monitor each beam pulse average energy, which has been delivered to the patient.

Such a highly responsive system is fast enough to allow the system to take actions to disable generation of the next beam pulse.

The system according to the invention makes no assumptions about the speed at which the beam energy can be changed, thus it poses no restrictions on the energy change rate. This is an improvement over the state-of-the-art because current beam energy measurement systems are either destructive or they do not allow the measurement of fast beam energy changes.

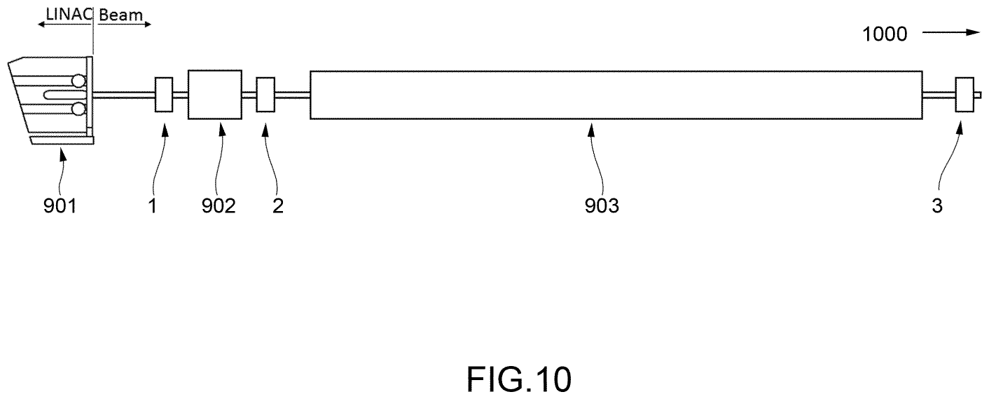

A particular embodiment of the invention is shown in FIG. 10, which shows a HEBT in which the invention may be situated. The last accelerating unit of the linear accelerator (901) issues the proton beam which comprises pulses, each of which comprises bunches, which bunches then pass the first detector (1), the second detector (2) and finally the third detector (3), all of which measure a bunch of the proton beam.

In a particular embodiment the last accelerating unit may be a CCL.

The detectors (1, 2, 3) share space in the HEBT with other components (902, 903) for example quadrupoles, ACCTs, BPMs, vacuum pumps, etc. The actual components present will depend on the particular HEBT layout, which will depend on the particular geometry of the installation.

The distances between the detectors are:

L₁₂=255 mm and

L₁₃=3595 mm.

These distances allow achievement of 0.03% E resolution for beams ranging from 70 up to 230 MeV, as shown in FIG. 2.

After passing through the third detector (3) the proton pulse will continue along the remainder of the HEBT (1000) which leads the beam towards a nozzle and then, finally, the patient placed in a treatment room.

### REFERENCES

1 first detector
2 second detector
3 third detector
5 amplifier
6 mixer and filter
7 FPGA
10 procedural step
20 procedural step
30 procedural step
40 procedural step
100 detection step
101 sampling step
102 calculation step
200 frequency calculation step
300 I/Q method step
301 I, Q signal calculation step
302 phase and amplitude calculation step
400 comparison step
500 phase shift calculation step
600 wrapping step
700 Energy value calculation step
701 calculation step
800 mean value calculation step

1. A time-of-flight (TOF) measurement system for measuring energy of a pulsed hadron beam, wherein each pulse of the beam is structured into a series of bunches (B) of charged particles, said bunches being repeated according to a repetition rate of the order of magnitude of radiofrequency, said system comprising
a first detector (1), a second detector (2) and a third detector (3) arranged along a beam path (10), each of said detectors being configured to detect the passage of a bunch (B) of charged particles and provide an output signal (_{νPP.1, νPP.2, νPP.3}) dependent on phase of the detected bunch (B), wherein the second detector (2) is spaced apart from the first detector (1) by a first distance (L₁₂) and wherein the third detector (3) is spaced apart from the second detector (2) by a second distance (L₂₃), wherein said first distance is set out in such a way as that time of flight (t₁₂) of the bunch (B) from the first detector (1) to the second detector (2) is approximately equal to, or lower than a repetition period (T_{RFQ}) of the bunches (B), and wherein said second distance is set out in such a way as that time of flight (T₂₃) of the bunch (B) from the second detector (2) to the third detector (3) is greater than a multiple of the repetition period (T_{RFQ}) of the bunches (B), and
processing means (7) configured to
a) calculate phase shifts (Δϕ₁₂, Δϕ₁₃, Δϕ₂₃) between the output signals (_{νPP.1, νPP.2, νPP.3}) of the detectors (1, 2, 3), and
b) calculate energy (E) of the pulse based on the calculated phase shifts.

2. A system according to claim 1, wherein said step a) comprises
detecting a frequency (f_{g}) of the output signals (_{νPP.1, νPP.2, νPP.3}) of the detectors (1, 2, 3), and
performing an I/Q method on the output signals (_{νPP.1, νPP.2, νPP.3}), based on the detected frequency (f_{g}), to calculate the amplitude (A_{PP.1}, A_{PP.2}, A_{PP.3}) and phase (ϕ_{PP.1}, ϕ_{PP.2}, ϕ_{PP.3}) of each output signal (_{νPP.1, νPP.2, νPP.3}).

3. A system according to claim 1, wherein said detectors are detectors responsive to
an electric field or magnetic field of the pulsed hadron beam passing thereby.

4. A system according to claim 1, wherein said detectors are detectors intercepting
a fraction of the pulsed hadron beam.

5. A system according to claim 1, wherein the repetition rate of the bunches
(B) is of an order of magnitude comprised between 100 MHz and 3 GHz, and preferably comprised between 100 MHz and 1 GHz.

6. Radiotherapy apparatus comprising at least one linear accelerator configured to
produce and accelerate a hadron beam, and further comprising a beam energy measurement system according to claim 1.

7. Apparatus according to claim 6, wherein said linear accelerator is configured to produce and accelerate a proton beam.

8. A time-of-flight (TOF) measurement method for measuring energy of a pulsed hadron beam, wherein each pulse of the beam is structured into a series of bunches (B) of charged particles, said bunches being repeated according to a repetition rate of the order of magnitude of radiofrequency,
wherein a first detector (1), a second detector (2) and a third detector (3) arranged along a beam path (10) are used, each of said detectors being configured to detect the passage of a bunch (B) of charged particles and provide an output signal (_{νPP.1, νPP.2, νPP.3}) dependent on phase of the detected bunch (B), wherein the second detector (2) is spaced apart from the first detector (1) by a first distance (L₁₂) and wherein the third detector (3) is spaced apart from the second detector (2) by a second distance (L₂₃), wherein said first distance is set out in such a way as that time of flight (t₁₂) of the bunch (B) from the first detector (1) to the second detector (2) is approximately equal to, or lower than a repetition period (T_{RFQ}) of the bunches (B), and wherein said second distance is set out in such a way as that time of flight (T₂₃) of the bunch (B) from the second detector (2) to the third detector (3) is greater than a multiple of the repetition period (T_{RFQ}) of the bunches (B), and
wherein said method comprises:
a) calculating phase shifts (Δϕ₁₂, Δϕ₁₃, Δϕ₂₃) between the output signals (_{νPP.1, νPP.2, νPP.3}) of the detectors (1, 2, 3), and
b) calculating energy (E) of the pulse based on the calculated phase shifts.
